(19) United States
(12) Patent Application Publication
(10) Pub. No.: US 2007/0218494 A1
(43) Pub. Date: Sep. 20, 2007

(54) Branched polymer lables as drag-tags in free solution electrophoresis

(76) Inventors: Gary Slater, Ottawa (CA); Sorin Nedelcu, Ottawa (CA)

Correspondence Address: KIRBY EADES GALE BAKER, BOX 3432, STATION D, OTTAWA, ON, K1P 6N9, CA

(21) Appl. No.: 11/723,321

(22) Filed: Mar. 19, 2007

Related U.S. Application Data
(60) Provisional application No. 60/783,034, filed on Mar. 17, 2006.

(51) Int. Cl. C12Q 1/68

(52) U.S. Cl. 435/6

(57) ABSTRACT

End Labelled Free Solution Electrophoresis (ELFSE) provides a means of separating polymer molecules such as ssDNA according to their size, via free solution electrophoresis, thus eliminating the need for polymer separation via gels or polymer matrices. Here, end labels are provided that optimize branching architecture to increase hydrodynamic drag of the end label, and improve separation of polymer molecules by ELFSE.

### CROSS-REFERENCE TO RELATED APPLICATION

This application claims the priority right of prior U.S. patent application No. 60/783,034 filed Mar. 17, 2006 by applicants herein.

### FIELD OF THE INVETION

The invention relates to the field of polymer separation. More particularly, the invention relates to the separation of polymer molecules of different sizes.

### BACKGROUND TO THE INVENTION

Techniques for separation of polymer molecules on the basis of their size are well known in the art. For example, polynucleotides or polypeptides may be separated via gel-based electrophoresis techniques, which involve gel matrices comprising for example agarose or polyacrylamide. In the case of DNA sequencing, polynucleotides may be separated with a resolution as low as a single polymer unit (nucleotide).

End Labelled Free Solution Electrophoresis (ELFSE) provides a means of separating DNA with free solution electrophoresis, eliminating the need for gels and polymer solutions. In free solution electrophoresis, DNA is normally free-draining and all fragments elute at the same time. In contrast, ELFSE often uses uncharged label molecules attached to each DNA fragment in order to render the electrophoretic mobility of the DNA fragments size-dependent. For example, methods for ELFSE are disclosed for example in U.S. Pat. Nos. 5,470,705, 5,514,543, 5,580,732, 5,624,800, 5,703,222, 5,777,096, 5,807,682, and 5,989,871, all of which are incorporated herein by reference. Many types and variations of end labels are known in the art, as described in the aforementioned patents, as well as U.S. patent publication US2006/0177840 published May 1, 2006, which is also incorporated herein by reference.

The nature of the end labels (also known as ‘drag-tags’) can vary significantly. Typically, an end label refers to any chemical moiety that may be attached to or near to an end of a polymeric compound to increase the drag of the complex during free solution electrophoresis, wherein the drag is caused by hydrodynamic friction. It is desirable to use end labels that induce a significant amount of hydrodynamic friction, since this may improve the ELFSE process. For example, end labels with significant hydrodynamic friction may permit greater separation of a larger range of polymer molecule sizes. When applied to DNA sequencing methods, this may translate into greater nucleotide resolution and/or increased read lengths.

In specific examples, a drag tag may comprise a peptide or a polypeptoid comprising up to or more than 100, preferably up to 200, more preferably up to or more than 300 polymer units. If required, the drag-tags or end labels may be uncharged such that they merely act to cause drag upon the charged polymeric compound during motion through a liquid substance.

There is a general desire in the art to produce end labels that are simple to manufacture, simple to attach to a polymer molecule, and which cause a significant degree of hydrodynamic drag in solution (when the end labeled polymer molecule is subjected for example to electrophoresis, optionally with an electroosmotic flow). However, the mechanisms that give rise to relative increases in hydrodynamic drag are poorly understood. It follows that there remains a need to develop further improved end labels and corresponding methods for polymer separation by optimization of the properties of the end labels. For example, there remains a need to develop methods for DNA sequencing via ELFSE with increased nucleotide sequence resolution and sequence read length. There is also a need to develop improved design rules to help optimize hydrodynamic drag properties of end labels.

### SUMMARY OF THE INVENTION

It is an object of the invention, at least in preferred embodiments, to provide a method for separating polymer molecules on the basis of their size.

It is another object of the invention, at least in preferred embodiments, to provide a method for sequencing DNA.

In one aspect the invention provides an end label suitable for attachment at or near to an end of a polymer molecule, so as to increase the hydrodynamic drag of the polymer molecule during motion through a liquid substance such as during electrophoresis, with or without the presence of an electroosmotic flow, the end label comprising:

(1) a backbone such as a substantially linear backbone;

(2) at least one branch arm extending from the backbone at branch point(s) therein, the branch arm(s) selected from at least one of the group consisting of:
(2a) a plurality of branch arms each being substantially shorter than the backbone and having a length about equal to or greater than a length of the substantially linear backbone between adjacent consecutive branch points; and
(2b) at least one branch arm each extending from a corresponding branch point at or near at least one end of the linear backbone, each branch arm optionally including further iterative branching extending from a free end thereof.

Preferably, the backbone comprises from 20-10000 monomer units.

Preferably, the branch arms of (2a) comprise from 2-1000 branch arms, each comprising from 5-10000 monomer units. Preferably, the branch arms of (2b) comprise from 2-1000 branch arms, each comprising from 5-10000 monomer units. Each backbone and/or each branch arm may be charged or uncharged.

Preferably, the substantially linear backbone and each branch arm each comprise monomer units. More preferably, the end label comprises from 30-500 monomer units. Preferably, the end label is a polypeptide and/or polypeptoid, and the monomer units comprise natural and/or non-natural amino acids.

Preferably, the at least one branch arm each extending from a corresponding branch point at or near at least one end of the linear backbone, comprises two branch arms each extending from an opposite end of the substantially linear backbone.

Preferably, the plurality of branch arms extending from the linear backbone at branch points therein are substantially equally spaced along the linear backbone, each branch arm having a length substantially equal to every other branch arm, and substantially equal to a length of said linear backbone between consecutive branch points.

Preferably, the at least one branch arm each extending from a corresponding branch point at or near at least one end of the linear backbone, each including iterative branching comprising at least two further branch arm extensions to each branch arm, each extension extending at or near an end of each previous extension closer to the substantially linear backbone.

In another aspect of the invention there is provided a method for constructing an end label for attachment to a polymer molecule to increase the hydrodynamic drag of the molecule through a liquid such as during electrophoresis or electroosmotic flow, the method comprising the steps of:

(1) synthesizing a substantially linear backbone comprising a plurality of monomer units; and

(2) synthesizing at least one branch arm extending from the backbone at branch point(s) therein, the branch arm(s) selected from at least one of the group consisting of:
(2a) a plurality of branch arms each being substantially shorter than the backbone and having a length about equal to or greater than a length of the substantially linear backbone between adjacent consecutive branch points; and
(2b) at least one branch arm each extending from a corresponding branch point at or near at least one end of the linear backbone, each branch arm optionally including further iterative branching extending from a free end thereof.

Preferably, the monomer units are natural and/or unnatural amino acids, said end label comprising a polypeptide and/or a polypeptoid.

In another aspect the invention provides a plurality of covalently modified polymer molecules having more than one length, suitable for separation via ELFSE, each comprising a substantially linear sequence of monomer units, and having covalently attached to at least one end thereof an end label of the invention. Preferably, each polymer molecule in the plurality of covalently modified polymer molecules comprises ssDNA, derived from at least one DNA sequencing reaction.

In another aspect of the invention there is provided a method for sequencing a section of a DNA molecule, the method comprising the steps of:

(a) synthesizing a first plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific adenine base in said section of DNA;

(b) synthesizing a second plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific cytosine base in said section of DNA;

(c) synthesizing a third plurality of ssDNA molecules each comprising a sequence identical to at least a portion at or near the 5′end of said section of DNA, said ssDNA molecules having substantially identical 5′ ends but having variable lengths, the length of each ssDNA molecule corresponding to a specific guanine base in said section of DNA;

(d) synthesizing a fourth plurality of ssDNA molecules each comprising a sequence identical to at least a portion at or near the 5′end of said section of DNA, said ssDNA molecules having substantially identical 5′ ends but having variable lengths, the length of each ssDNA molecule corresponding to a specific thymine base in said section of DNA;

(e) attaching an end label of claim 1 at or near at least one end of said ssDNA molecules to generate end-labeled ssDNAs; and

(f) subjecting each plurality of end labelled ssDNA molecules to free-solution electrophoresis;

(g) identifying the nucleotide sequence of the section of DNA in accordance with the relative electrophoretic mobilities of the end labeled ssDNAs in each plurality of ssDNAs;

wherein any of steps (a), (b), (c), and (d) may be performed in any order or simultaneously;

whereby each end label imparts increased hydrodynamic friction to at least one end of each end-labeled ssDNAs thereby to facilitate separation of the end-labeled ssDNAs according to their electrophoretic mobility.

Preferably, the section of DNA comprises less than 2000 nucleotides, more preferably less than 500 nucleotides, more preferably less than 100 nucleotides.

In another aspect the invention also provides a DNA sequencing kit comprising the end label of claim 1, together with at least one other component for a DNA sequencing reaction.

### BRIEF DESCRIPTION OF THE DRAWINGS

FIG. 1 schematically illustrates a representation of the blob theory of ELFSE. The linear drag-tag (dotted line) is attached at one end to the linear ssDNA molecule (dark line). We construct blobs of identical hydrodynamic radii in order to take into account the differences in the persistence length (stiffness) and monomer size between the two polymers. These blobs will act as super-monomers in our theory.

FIG. 2 schematically illustrates a representation of the chemical nature of the backbone monomers of the drag-tags; the bond lengths from one alpha carbon to the next along the backbone are 0.151 nm, 0.1325 nm and 0.1455 nm, giving a total monomer size of about 0.43 nm.

FIG. 3 schematically illustrates a drawing of a branched label with a =4 arms, uniformly spaced along the backbone, N₁ monomers along each arm, and N_{b} monomers between arms. The two end segments have N_{b}′ monomers each. Letters A to D indicate the possible relative positions of any two monomers (see text).

FIG. 4 schematically shows a ssDNA molecule attached to a 3-branch drag-tag molecule; (a) in the large blob theory of ELFSE the drag-tag forms one blob, (b) in the small blob theory the three branching points are the centres of the three blobs.

FIG. 5 provides a schematic definition of the branching points. (a) The two monomers A and B are separated by two KP chains and one branching point. (b) The two monomers A and B are separated by three KP chains and two branching points.

FIG. 6 provides a schematic representation of two Kratky-Porod (KP) chains that connect monomer A and monomer B. The two KP chains are of lengths n₁ and n-n₁, respectively.

FIG. 7 shows predictions of WLC model in the presence of branching points for the a values of the a) tetramer and b) octamer labels. The cases where the branching angles δ are identical to the average bond angle θ of the backbone, or side chains, are also indicated on the figures.

FIG. 8 illustrates the relative difference between the hydrodynamic radii of a linear (R_{I}) and of a branched (R_{H}) drag-tag (FIG. 3) is plotted as function of the length N₁ of the arms of the branched molecule. In this case, both polymers are assumed to be freely jointed chains.

FIG. 9 illustrates the relative difference between the hydrodynamic radii of a linear ( R_{I}) and of a branched (R_{H}) drag-tag (FIG. 3) as function of the length N₁ of the arms of the branched molecule. In this case, both polymers are assumed to be worm-like chains with a persistence length of lₚ0.39 nm and a corresponding branching angle of δ=θ=70.6°

FIG. 10 illustrates the hydrodynamic radius of branched labels made of a fixed total number of monomers N: a) N=50 and b) N=70. The size of the free ends was set to N_{b}′=3 in all cases. Each curve is for a different number a of arms. Since the x-axis gives the length N₁ of the arms, the last parameter (the distance N_{b} between the arms along the backbone) is given by the equation N =a(N_{b}+N₁)+2N_{b}′−N_{b}; the numbers are given in the legend for each data point on the graphs (counting from left to right). For the 50-mer and 70-mer labels, the experimental data correspond to points (N₁=4, N_{b}=6, a=5) and (N₁=8, N_{b}=6, a=5) respectively. The hydrodynamic radii of the corresponding linear molecules would be R_{H}(N=50)=1.27 nm and R_{H}(N=70)=1.46 mn.

FIG. 11 schematically illustrates preferred branching arrangements or patterns for particularly preferred end labels of the present invention, which confer particularly useful degrees of hydrodynamic drag to polymer molecules to which they are attached. (a) illustrates an end label comprising a substantially linear backbone, with a plurality of short branch arms having a length about equal to a distance between branch points of the branch arms along the backbone; (b) illustrates an end label comprising a substantially linear backbone, with a longer branch arm at each end of the backbone; (c) illustrates an end label as per FIG. 11 (b) further comprising iterative branching of the branch arms.

### DEFINITIONS

‘Branch point’ refers to a point in a backbone portion of an end label, or a point in a branch arm, at which a branch arm (or further branch arm) commences. In this way, a branch point is a point of intersection of a branch arm of an end label with either a backbone portion or another branch arm of the end label.
‘Branched’—refers to there being at least one branch of monomer units in a polymer compound or an end label comprising monomer units. ‘Branched’ may refer only to the presence of a single branch, or alternatively to multiple branches or branches of branches. Moreover, branching may be iterative such that a branch may itself be branched.
‘Drag’—whether used as a noun or as a verb, ‘drag’ refers to impedance of movement of a molecule through a viscous environment (such as an aqueous buffer), such as for example during electrophoresis, either in the presence or the absence of a sieving matrix. More typically, ‘Drag’ refers to ‘hydrodynamic drag’ as will be understood by a person of skill in the art, particularly one who has read and understood the foregoing.
ELFSE—End Labeled Free Solution Electrophoresis. The preferred conditions for ELFSE are apparent to a person of skill in the art upon reading the present disclosure, and the references cited herein
EOF—electroosmotic flow.
‘End label’ or ‘Label’ or ‘tag’ or ‘drag-tag’: refers to any chemical moiety that may be attached to or near to an end of a polymeric compound to increase the drag of the complex during free solution electrophoresis, wherein the drag is caused by hydrodynamic friction. In selected examples, the drag tag may comprise a linear or branched peptide or a polypeptoid comprising up to or more than 10000, preferably up to 1000 polymer units, Each tag or label may take any form of sufficient configuration or size to cause a sufficient degree of drag during free-solution electrophoresis and/or EOF. For example each label or tag may be a substantially linear, alpha-helical or globular polypeptide comprising any desired amino acid sequence. Moreover, each label or tag may comprise any readily available protein or protein fragment such as an immunoglobulin or fragment thereof, Steptavidin, or other protein generated by recombinant means. In a preferred embodiment each label or tag may be a polypeptoid comprising a linear or branched arrangement of amino acids or other similar units that do not comprise L-amino acids and corresponding peptide bonds normally found in nature. In this way the polypeptoid may exhibit a degree of resistance to degradation under experimental conditions, for example due to the presence of proteinases such as Proteinase K. Preferably, the tags or labels are not charged such that they merely act to cause drag upon the charged polymeric compound during motion through a liquid substance. However, the invention is not limited in this regard, and the present specification teaches the use of charged tags or labels. The invention further teaches optimal branch patterns for the end label, as will be clarified by the foregoing. Each end label or drag tag may further include a further tag or label such as a flurescent tag or label for use in identifying the end label or drag tag, such as for example during automated DNA sequencing.
‘Linear’—refers to a length of a polymer molecule, or a length of a portion of an end label comprising monomer units, in which there are no or substantially no branches of further monomer units. ‘Linear’ does not preclude the option that branches may be present, and therefore may be used for example to refer to a ‘linear backbone’ of a polymeric structure of an end label. ‘Linear’ does not necessarily require that the A backbone be straight, but rather specifies a general absence of branches or other delineations.
MALDI-TOF—matrix-assisted laser desorption/ionization time-of-flight;
‘Near’—In selected embodiments of the invention end labels are described herein as being attached at or near to each end of a polymeric compound. In this context the term ‘near’ refers to attachment of a tag or chemical moiety to a monomeric unit of a polymer molecule in the vicinity of an end of the polymer molecule. Alternatively, the term ‘near’ may refer to a branched arm of an end label extending in the vicinity of the end of a branch arm of the polymer molecule. In addition, the term “near” may vary in accordance with the context of the invention, including the size and nature of the moiety or tag, or the length and shape of the polymer molecule. For example, in the case of a short polynucleotide comprising less than 20 bases, the term “near” may, for example, preferably include those nucleotides within 5 nucleotides from each end of the polynucleotide. However, in the case of a longer polynucleotide comprising more than 100 bases then the term “near” may, for example, include those nucleotides within 20-100 nucleotides from each end of the polynucleotide. Typically, “near” can mean within 25%, preferably 15%, more preferably 5% of an end of a polymer molecule relative to an entire length of the polymer molecule;
PEG—poly(ethylene glycol);
‘Polymer molecule’—refers to any polymer whether of biological or synthetic origin, that is linear or branched and composed of similar if not identical types of polymer units. In preferred embodiments, the polymer molecules are linear, and in more preferred embodiments the polymeric compounds comprise nucleotides or amino acids. The polymer molecule is preferably a polypeptide or a polynucleotide. More preferably the polymer molecule is a polynucleotide and the method of the present invention is suitable to separate the polynucleotide from other polynucleotides of differing size. Moreover, the polynucleotide may comprise any type of nucleotide units, and therefore may encompass RNA, dsDNA, ssDNA or other polynucleotides. In a more preferred embodiment of the invention, the polymer molecule is ssDNA, and the methods permit the separation of compounds that are identical with the exception that the compounds differ in length by a single nucleotide or a few nucleotides. In this way the methods of the present invention, at least in preferred embodiments, permit the separation and identification of the ssDNA products of DNA sequencing reactions. The size of the tag or label positioned at one or each end of the ssDNA molecules is (at least in part) a function of the read length of the DNA sequencing that one may want to achieve. With increasing size or hydrodynamic drag of labels or tags the inventors expect the methods of the present invention to be applicable for sequencing reactions wherein a read length of up to 2000 nucleotides is achieved. With other tags or labels shorter read length may also be achieved including 300, 500, or 1000 base pairs. The desired read lengths will correspond to the use to which the DNA sequencing is applied. For example, analysis such as single nucleotide polymorphism (SNP) analysis may require a read length as small as 100 nucleotides, whereas chromosome walking may require a read length as long as possible, for example up to 2000 base pairs.
‘Polypeptoid’—a linear or non-linear chain of amino-acids that comprises at least one non-natural amino acid that is not generally found in nature. Such non-natural amino acids may include, but are not limited to, D-amino acids, or synthetic L-amino acids that are not normally found in natural proteins. In preferred embodiments, polypeptoids are not generally susceptible to degradation by proteinases such as proteinase K, since they may be unable to form a protease substrate. In selected embodiments, polypeptoids may comprise exclusively non-natural amino acids. In further selected embodiments, polypeptoids may typically but not necessarily form linear or alpha-helical (rather than globular) structures.
‘Preferably’ and ‘preferred’—make reference to aspects or embodiments of the inventions that are preferred over the broadest aspects and embodiments of the invention disclosed herein, unless otherwise stated.

### DETAILED DESCRIPTION OF THE PREFERRED EMBODIMENTS OF THE INVENTION

Polymeric compounds, such as polypeptides and polynucleotides, are routinely subject to modification. Chemical synthesis or enzymatic modification can enable the covalent attachment of artificial moieties to selected units of the polymeric compound. Desirable properties may be conferred by such modification, allowing the polymeric molecules to be manipulated more easily. In the case of DNA, enzymes are commercially available for modifying the 5′ or 3′ ends of a length of ssDNA, for example to phosphorylate or dephosphorylate the DNA. In another example, biotinylated DNA may be formed wherein the biotin moiety is located at or close to an end of the DNA, such that Strepavidin may be bound to the biotin as required. Tags such as fluorescent moieties may also be attached to polynucleotides for the purposes of conducting DNA sequencing, for example using an ABI Prism™ sequencer or other equivalent sequencing apparatus that utilizes fluorimetric analysis.

In the framework of the classical blob theory of End-Labeled Free Solution Electrophoresis (ELFSE) of ssDNA and other polymer molecules, and based on recent experimental data with linear and branched polymeric labels (or drag-tags), the present invention provides design principles for the optimal type of branching that would give, for a given total number of monomers, the highest effective frictional drag for example for ssDNA sequencing purposes. The hydrodynamic radii of the linear and branched labels are calculated using standard models like the freely jointed chain model and the Kratky-Porod worm like chain model.

To separate DNA fragments by free solution electrophoresis is an impossible task [1], unless the free-draining DNA polymer is modified at the molecular level, e.g. by conjugation with an uncharged “drag” molecule that can change its hydrodynamic friction without affecting its total charge [2]. The charge-to-friction ratio then becomes a function of the DNA chain length and free solution electrophoresis becomes possible. The conjugation method has been applied successfully to separate ssDNA fragments up to a maximum length of ≈120 bases, with single monomer resolution [3]. The method has been called End-Labeled Free Solution Electrophoresis (ELFSE) [4]. The key parameter of the ELFSE method is clearly the effective hydrodynamic friction provided by the drag-tag. To successfully apply ELFSE for ssDNA sequencing, we need to maximize the value of this fundamental property [5-7]. Given all the constraints that ELFSE is working under, this is not an easy task. For instance, simply increasing the length of a water-soluble and neutral linear polymeric drag-tag is not as easy as it seems because the drag-tags must also remain perfectly monodisperse (i.e., to within one monomer). An alternative, recently proposed by Haynes et at. (Bioconjugate Chem 2005, 16, 929-938) is to use branched polymers with a fixed architecture. The present invention extended this work significantly to provide ways to optimize such a branched structure for ELFSE.

Since the theory of ELFSE is rather well documented [8, 9], we will use the classical model (described in Example 1) to analyze the recent experimental data (presented in Example 2 and 3) with linear and branched labels (Haynes et al.,). In order to compute the effective hydrodynamic radius of the various branched labels, we will first use the freely jointed chain (FJC) model and the equations derived by Teraoka [10] for branched FJC polymers. As we shall see, the predicted friction coefficients will be too small to explain the experimental data (Example 4-8). This indicates that a more detailed treatment of a branched worm like chain (WLC) model is necessary. We develop two such models in Section 5: in the first case, we take into account the finite persistence length of the polymer, but we disregard the branching points; in the second approach, we also consider the effect of the branching points.

In fact, it is possible to predict the hydrodynamic radius of branched drag-tags, what remains is a constrained optimization problem that can be phrased in the following way: What is the best strategy to distribute a set number of monomers onto primary (or even secondary) side chains such that we maximize the drag-tag's effective ELFSE friction coefficient? Corresponding aspects of the invention, as well as general branching strategies for drag-tags for use in ELFSE, will also be shown.

In preferred embodiments the invention encompasses an end label suitable for attachment at or near to an end of a polymer molecule, so as to increase the hydrodynamic drag of the polymer molecule during motion through a liquid substance such as during electrophoresis, with or without the presence of an electroosmotic flow, the end label comprising:

(1) a backbone such as a substantially linear backbone;

(2) at least one branch arm extending from the backbone at branch point(s) therein, the branch arm(s) selected from at least one of the group consisting of.
(2a) a plurality of branch arms each being substantially shorter than the backbone and having a length about equal to or greater than a length of the substantially linear backbone between adjacent consecutive branch points; and
(2b) at least one branch arm each extending from a corresponding branch point at or near at least one end of the linear backbone, each branch arm optionally including further iterative branching extending from a free end thereof.

Preferably, the backbone comprises from 20-10000 monomer units.

Preferably, the branch arms of (2a) comprise from 2-1000 branch arms, each comprising from 5-10000 monomer units. Preferably, the branch arms of (2b) comprise from 2-1000 branch arms, each comprising from 5-10000 monomer units. Each backbone and/or each branch arm may be charged or uncharged. However, the number of monomer units in the backbone or any branch arm, or the number of branch arms, may vary even further in accordance with the end labels of the present invention, providing the desired attributes for the end label of superior levels of hydrodynamic drag are exhibited.

In more preferred embodiments each end label comprises from 30-500 monomer units. The monomer units may be derived from or form any polypeptide and/or polypeptoid, and the monomer units may comprise natural and/or non-natural amino acids.

Particularly preferred configurations, positions, and lengths for the branch arms, which provide particularly increased levels of hydrodynamic drag, will be apparent from the present discussion.

In further preferred embodiments the invention provides methods for constructing an end label for attachment to a polymer molecule to increase the hydrodynamic drag of the molecule through a liquid such as during electrophoresis or electroosmotic flow, the methods comprising the steps of:

(1) synthesizing a substantially linear backbone comprising a plurality of monomer units; and

(2) synthesizing at least one branch arm extending from the backbone at branch point(s) therein, the branch arm(s) selected from at least one of the group consisting of:
(2a) a plurality of branch arms each being substantially shorter than the backbone and having a length about equal to or greater than a length of the substantially linear backbone between adjacent consecutive branch points; and
(2b) at least one branch arm each extending from a corresponding branch point at or near at least one end of the linear backbone, each branch arm optionally including further iterative branching extending from a free end thereof.

In still further embodiments the invention provides a plurality of covalently modified polymer molecules having more than one length, suitable for separation via ELFSE, each comprising a substantially linear sequence of monomer units, and having covalently attached to at least one end thereof an end label of the invention. Preferably, each polymer molecule in the plurality of covalently modified polymer molecules comprises ssDNA, derived from at least one DNA sequencing reaction.

Particularly preferred embodiments of the invention provide a method for sequencing a section of a DNA molecule, the method comprising the steps of:

conducting a sequencing reaction for a length of DNA using labelled chain terminator nucleotides to form ssDNAs;

attaching an end label of the invention at or near at least one end of said ssDNA molecules to generate end-labeled ssDNAs; and

subjecting each plurality of end labelled ssDNA molecules to free-solution electrophoresis;

identifying the nucleotide sequence of the section of DNA in accordance with the relative electrophoretic mobilities of the end labeled ssDNAs in each plurality of ssDNAs;

whereby each end label imparts increased hydrodynamic friction to at least one end of each end-labeled ssDNAs thereby to facilitate separation of the end-labeled ssDNAs according to their electrophoretic mobility.

In preferred embodiments such methods may permit sequencing of up to or even more than a read length of 2000 nucleotides.

The following examples illustrates preferred embodiments of the invention, and are in no way intended to be limiting to the invention disclosed and claimed herein:

### EXAMPLE 1

### ELFSE for Linear Drag-Tags

Meagher et al. [2] have recently reviewed the evolution of ELFSE over the last decade, including the theoretical concepts used to analyze experimental data and the technological progress still needed to develop a competitive ELFSE-base sequencing method. Although the exact conformation of the composite ssDNA/drag-tag molecule is in principle important for deriving accurate ELFSE theories, we shall assume in the following that there is no physical segregation of the ssDNA and the label. We shall also assume that the label is not deformed, which means that the hybrid molecule is globally a random coil of effective hydrodynamic blobs. Previous studies indicated that these two assumptions can indeed explain currently available data. For the sake of completeness, we now review the corresponding theoretical arguments.

The electrophoretic mobility μ of a block copolymer consisting of a linear chain of M_{c} charged monomers linked to a linear chain of Mₙ uncharged but otherwise identical monomers has been shown [11- 13] to be given by the following relation:

$$μ = {μ}_{0} \times \frac{{M}_{c}}{{M}_{c} + {M}_{u}} \qquad ( 1 )$$

where μ₀ is the free electrophoretic mobility—without the drag-tag—of the charged polymer. This equation neglects the correction due to the effects of the ends of the molecule [9].

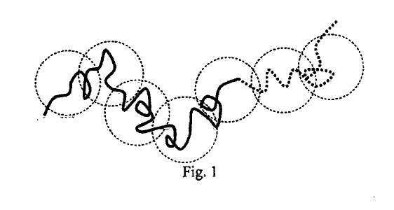

Arguing that uncharged label monomers are not always equivalent to ssDNA monomers from a hydrodynamic point of view, and therefore that a non-uniform weighted average of the monomers' mobilities should be used, McCormick et al. [8] developed the blob theory of ELFSE (FIG. 1). Since the monomers are not equivalent, they are regrouped into blobs of identical properties; these blobs then act as super-monomers, and one can use Eq. (1) to describe their global ELFSE behavior. This theory thus replaces Eq. (1) by the relation:

$$\begin{matrix}μ = {μ}_{0} \times \frac{{M}_{c}}{{M}_{c} + {α}_{1} {M}_{u}} = \frac{{μ}_{0}}{1 + \frac{α}{{M}_{c}}} where & ( 2 a ) \\ {α}_{1} ≡ \frac{{b}_{u} {b}_{Ku}}{{b}_{c} {b}_{Kc}} & ( 2 b )\end{matrix}$$

is a dimensionless parameter, bᵤ and b_{c} are the monomer sizes of the charged and uncharged monomers respectively, and b_{Ku} and b_{Kc} are the corresponding Kuhn lengths. The Kuhn statistical segment length is a measure of polymer stiffness, and can be calculated from the local structure of the chain. It can be defined by b_{K}≡ [symbol] R² [symbol] /Rₘₐₓ, where R is the chain's end-to-end distance and Rₘₐₓ is its maximum value. Actually, Eq. (2b) was derived using this definition and assuming that both polymer chains are much longer than their Kuhn lengths. Note that for a perfectly flexible molecule (such as a FJC), one has b_{K}=b, Rₘₐₓ=Mb and [symbol] R² [symbol] =Mb². The definition of the Kuhn length means that a stiff polymer can be treated as a FJC made of N_{K}=Mb/b_{K} segments of length b_{K}.

Parameter a₁ , in Eq. (2b) is a relative friction coefficient and has no dimensions. In fact, a≡a₁Mᵤ is the number of ssDNA monomers required to form a molecule with a hydrodynamic radius equal to the hydrodynamic radius of the Mᵤ label monomers. Since ssDNA is generally stiffer than the polymers used as drag-tags a₁ is often much smaller than unity.

For an elution length L, the elution time of a labeled ssDNA fragment is given by:

$$t = \frac{L}{μE} = \frac{L}{{μ}_{0} E} ( 1 + {α}_{1} \frac{{M}_{n}}{{M}_{c}} ) \qquad ( 3 )$$

where E is the applied electric field. From Eq. (3) the total effective friction coefficient a═a₁Mᵤ specific to a drag-tag can be simply obtained from the slope of a plot of the reduced elution time t/(L/μE) vs. the inverse of the number of charged monomers 1/M_{c}.

### EXAMPLE 2

### Experimental Analysis of Linear Drag Tags

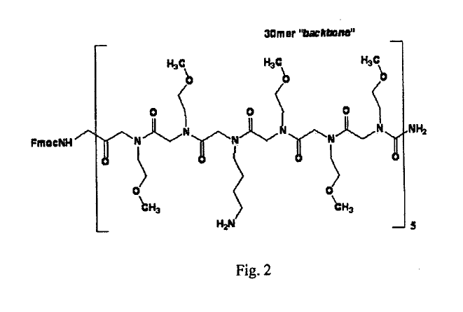

Haynes et al. first measured the electrophoretic migration times of unconjugated “free” DNA and of DNA conjugated to a linear drag-tag with Mᵤ=30 monomers. Using the equations derived in the previous section it is easy to compute the value of a₁ (or of the total effective drag coefficient a=a₁Mᵤ) from the two elution times thus measured. These authors repeated the experiments using M_{c}=20 as well as M_{c}=30 base ssDNA primers (Table 1). We note that both ssDNA molecules give the same result a=7.9 (equivalent to an effective drag coefficient of a₁=0.26 per uncharged drag-tag monomer). Equation (2b) can then be used to estimate the Kuhn length of this polymeric drag-tag: with b_{c}=0.43 nm and b_{Kc}=3 nm for ssDNA, and bᵤ=0.43 nm (estimated from the chemical structure, see FIG. 2) for the label, we obtain b_{Ku}=0.78 nm. This indicates that the label is very flexible: its Kuhn length is about twice its monomer size.

### EXAMPLE 3

### Experimental Analysis of Branched Drag-tags

In order to increase the effect of the drag-tag on the resolving power of ELFSE, one must build drag-tags with very large effective friction coefficients. Haynes et al. examined the role that branching could play in this process. To that end, they added branches to their initial Mᵤ=30 linear drag-tag. Using the equations of Example 2, they found that the apparent value of a increases roughly linearly with the molecular size of the branched label and the two ssDNA primers give slightly different values of a (see Table 1). Both of these results are surprising, and in selected embodiments the invention examines the physics that is relevant in the case of branched drag-tags.

TABLE 1
Experimental data for the linear (acetylated) and branched labels.
The experimental value of the drag-tag effective friction coefficient
α was obtained using the theory for linear labels presented
in Section 2 (Reproduced from Haynes et al.).

| | | 20 base DNA | 30 base DNA |
|---|---|---|---|
| | molar mass | Primer | Primer |
| Drag-tag | calculated:found | Total Drag (α) | Total drag (α) |
| Linear: 30-mer | 4023.2:4023.5 | 7.9 | 7.9 |
| Branched (tetramer): | 6964.9:6964.6 | | |
| 50-mer | | 12.5 | 13.7 |
| Branched (octamer): | 9266.1:9271.4 | | |
| 70-mer | | 16.4 | 17.2 |

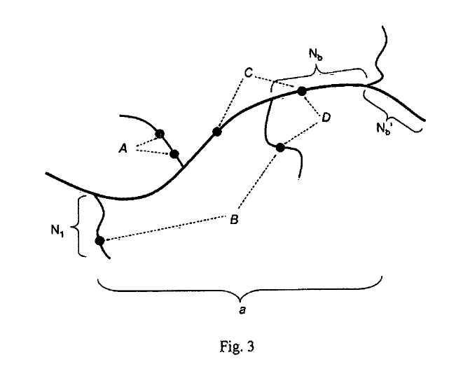

The terminology in Table 1 refers to a series of polypeptoid drag-tags based on a fixed thirty-residue “backbone” with branches forming stable amide bonds with the amino side-chains on the backbone. FIG. 2 presents a schematic representation of the backbone monomer unit. Depending on the number of monomers on the side chains, the labels were conventionally called the tetramer (for a total of 50 monomers) or the octamer (total of 70 monomers) branched labels. Both types have the same number of arms a=5 and the same number of bonds between arms N_{b}=6, but they possess different arm lengths, N₁=4 and N₁=8, respectively. FIG. 3 shows a schematic representation of these branched labels. The two end segments of the branched polymers have N_{b}′ monomers each (in the case of Haynes et al. N_{b}′=3).

### EXAMPLE 4

### Branched Freely-jointed Chain Polymers

As mentioned previously, Haynes et al. analyzed their data using the theory for linear labels (i.e., Eq. 2a). This theory applies in the case where the blob construction [8] is valid. However, it is not clear that this can be directly applied to the branched label.

In order to generalize the ELFSE theory to the case of branched labels, the inventors have determined how such hybrid molecules will be represented by blobs of identical hydrodynamic radii. There are two obvious ways to do this.

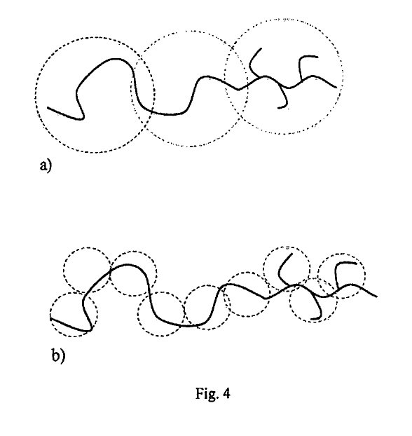

First, one can use the approach previously used for the bulky streptavidin label [2]: the whole label is seen as one uncharged blob (with a hydrodynamic radius R_{H}), and the ssDNA molecule is subdivided into blobs with the same size R_{H} (see FIG. 4a). The number m, of ssDNA monomers needed to build one such blob is simply given by a, and Eq. 2a can be used directly. Using this theory then simply requires that we compute the hydrodynamic radius R_{H} of the label and the value of m_{c}=m_{c}(R_{H}) using the proper model for the ssDNA.

The second approach, shown in FIG. 4b, consists in building a blob around each branching point. Each blob thus contains mᵤ(≅N_{b}+N₁) monomers, and the label is made of Mᵤ/mᵤ such uncharged blobs, each of radius r_{H}. The number m_{c} of ssDNA monomers needed to build one such blob must be found using a model for the hydrodynamic radius of both the DNA and the branched section of the label. Again, Eq. 2a can be used, but here Eq. 2b must be replaced by:

$${α}_{1} ≡ \frac{{m}_{c}}{{m}_{u}} \qquad ( 4 )$$

In this expression, mᵤ≅N_{b}+N₁ is known from the chemical structure while m_{c}=m_{c}(r_{H}(mᵤ)) must be computed. However, our drag-tags are so small that any Gaussian approximation would necessarily fail for the small blob model. We will thus focus our attention on the big blob model in subsequent examples. Note that in the Gaussian limit, and without excluded volume effects, one should have R_{H}²≅ar_{H}², and the two models should give the same answer.

In the example 5-7 we will examine the hydrodynamic radii of branched polymers in order to estimate the radius R_{H} of the whole label treated as a large blob (as shown in FIG. 4a) within our ELFSE theory.

### EXAMPLE 5

### Hydrodynamic Radii of Linear Freely Jointed Chains

The Kirkwood's approximation can be used to calculate the friction coefficient, or the Stokes hydrodynamic radius R_{H} of macromolecules:

$${R}_{H} = \frac{{N}^{2}}{2 {\sum }_{j } > {\sum }_{l} 〈 {R}_{ij}^{- 1} 〉} \qquad ( 5 )$$

where Rᵢⱼ is the distance between monomers i and j (the double sum is taken over all pairs of monomers). The simplest macromolecule is a linear chain of monomers with no correlation between the directions of the different bond vectors; this is generally called the Freely-Jointed Chain model (FJC). The average distance Rᵢⱼ between the i-th and the j-th segments of a FJC chain is given by: [symbol]
Rᵢⱼ² [symbol] =|i−j|b² (6)
Using the preaverage Eq. 6, the definition Eq. 5, and taking the sum over the pairs of monomers we obtain the hydrodynamic radius for a linear freely-jointed chain polymer:

$${R}_{H}^{2} = \frac{3 π}{128} {Nb}^{2} = \frac{3 π}{128} Lb \qquad ( 7 )$$

where L=Nb is the contour length of the linear FJC molecule. We note that R_{H}˜N^{1/2}, a standard result for random-walk models.

### EXAMPLE 6

### Hydrodynamic Radii for Branched Freely Jointed Chains

In a paper on the calibration of retention volumes in size exclusion chromatography, Teraoka recently derived an analytical expression for the hydrodynamics radius of a FJC branched polymer without excluded volume interactions. We shall use Teraoka's approach as this represents the simplest possible way to understanding the hydrodynamic properties of branched drag-tags. In our case, the total number of monomers is given by N=a(N₁+N)+2N_{b}′−N_{b}, where a is the number of arms along the backbone, N₁ is the number of bonds on each side chain, N_{b} is the number of bonds between the branched arms along the main backbone, and N_{b}′ is the number of bonds at each of the two ends of the molecule (see FIG. 3). Calculating R_{H} for this case requires that one properly calculates the mean distance between all pairs of monomers (see Eq. 5). Since a monomer is either on a side chain or on the backbone, we can distinguish the following four cases: (A) the two monomers are on the same side chain; (B) the two monomers are on different side chains; (C) the two monomers are on the chain's backbone; (D) one monomer is on the backbone while the other is on a side chain. In all four cases, the continuous sequence of monomers between the two monomers (the i,j pair in Eq. 5) being considered is treated as a FJC since excluded volume interactions are neglected in this model. Teraoka considered only the case where N_{b}′=N_{b}. We generalize his result to the case N_{b}′≠N_{b} and obtain: [symbol]
R_{H}⁻¹ [symbol] =C_{A} [symbol] R_{H}⁻¹ [symbol] _{A}+C_{B} [symbol] R_{H}⁻¹ [symbol] _{B}+C_{C} [symbol] R_{H}⁻¹ [symbol] _{C}+C_{D} [symbol] R_{H}⁻¹ [symbol] _{D} (8)
where
r=N₁/N_{b}, (8a)
C_{A}=aN₁²/N², (8b)
C_{B}=a(a−1)N₁²/N², (8c)
C_{C}=((a−1)N_{b}+2N_{b}¹)²/N², (8d)
C_{D}=2aN₁((a−1)N_{b}+2N_{b}¹)/N², (8e)
and

$$\begin{matrix}{〈 {R}_{H}^{- 1} 〉}_{A} = {( \frac{6}{π} )}^{1 / 2} \frac{8}{3} {N}_{1}^{- 1 / 2} \frac{1}{b} & ( 8 f ) \\ \begin{matrix}{〈 {R}_{H}^{- 1} 〉}_{B} = {( \frac{6}{π} )}^{1 / 2} \frac{8}{3 a ( a - 1 )} {r}^{- 3 / 2} {N}_{1}^{- 1 / 2} \frac{1}{b} {\sum }_{i = 1}^{a - 1} ( a - i ) [ {( 2 r + i )}^{3 / 2} - \\ 2 {( r + i )}^{3 / 2} + {i}^{3 / 2} ]\end{matrix} & ( 8 g ) \\ {〈 {R}_{H}^{- 1} 〉}_{C} = {( \frac{6}{π} )}^{1 / 2} \frac{8}{3} {( ( a - 1 ) {N}_{b} + 2 {{N}_{b}}^{′} )}^{- 1 / 2} \frac{1}{b} & ( 8 h ) \\ \begin{matrix}{〈 {R}_{H}^{- 1} 〉}_{D} = {( \frac{6}{π} )}^{1 / 2} \frac{8}{3 {aN}_{1} ( ( a - 1 ) {N}_{b} + 2 {{N}_{b}}^{′} )} \frac{1}{b} \times \\ \{ \begin{matrix}{\sum }_{l = 1}^{a - 1} [ {( {iN}_{b} + {N}_{1} )}^{3 / 2} - {( {iN}_{b} )}^{3 / 2} - {N}_{1}^{3 / 2} ] + \\ {( {N}_{1} + ( a - 1 ) {N}_{b} + {{N}_{b}}^{′} )}^{3 / 2} - \\ {( ( a - 1 ) {N}_{b} + {{N}_{b}}^{′} )}^{3 / 2} - {N}_{1}^{3 / 2}\end{matrix} \} \\ \end{matrix} & ( 8 i )\end{matrix}$$

The above expressions are simple functions of the various lengths, the number a of arms and the ratio r. We note that Eq. 8 reduces to Eq. 7 when N₁0.

### EXAMPLE 7

### Estimating a: Flexible Drag-Tags

In Section 3 we calculated the backbone monomer size from the bond lengths (see FIG. 2) and obtained the value of bᵤ≅0.43 nm. Since the arms' monomers are quite similar to the backbone monomers, we shall assume that they are identical, for simplicity. Since the precise structure of the drag-tags are known, their hydrodynamic radii can be predicted using Eqs. (8).

For the tetramer label we have the parameters: N₁=4, a=5, N_{b}=6, N_{b}′=3, and therefore, by substituting these values into Eqs. (8) we obtain R_{H}⁽⁵⁰⁾=0.70 nm. For the octamer-branched label the parameter N₁=8 is different and we obtain R_{H}⁽⁷⁰⁾=0.80nm instead. In order to make comparisons with the experiments, we need to convert the hydrodynamic radii into the corresponding effective drag-tag friction coefficients at. To this end, we need a model for ssDNA.

If we first assume that the ssDNA is also a flexible FJC polymer with a monomer size b_{c}=0.43 nm, we can use Eq. 7 to obtain

$$α = \frac{128}{3 π} \times \frac{{R}_{H}^{2}}{{b}_{c}^{2}} \qquad ( 9 )$$

where we simply wrote N=a in Eq. 7. For the tetramer and octamer labels, we then obtain a⁽⁵⁰⁾=36 and a⁽70)=47. These values are way too large, and they are also meaningless since one must take into account the stiffness of the ssDNA, which is a very rigid polymer.

For a sufficiently long linear stiff molecule (such as ssDNA), the radius of gyration of the coil is given by:

$${R}_{g}^{2} = \frac{1}{6} L {b}_{Kc} \qquad ( 10 )$$

where L=M_{c}b_{c} is the contour length of the polymer, b_{c} is the monomer length and b_{Kc} is the Kuhn length. Note that this expression neglects the effects of the excluded volume interactions. The relation between the radius of gyration R_{g} and the hydrodynamic radius R_{H} is approximately [14]:

$${R}_{H} ≅ \frac{2}{3} {R}_{g} \qquad ( 11 )$$

From Eqs. (10) and (11), we can thus write the hydrodynamic radius of the linear polymer:

$${R}_{H} ≅ \frac{2}{3 \sqrt{6}} \sqrt{{b}_{c} {b}_{Kc}} {M}_{c}^{1 / 2} \qquad ( 12 )$$

Using this expression, we can replace Eq. (9) by the more realistic relation

$$α {M}_{u} ≅ \frac{27}{2} ⨯ \frac{{R}_{H}^{2}}{{b}_{c} {b}_{Kc}} \qquad ( 13 )$$

Using the values mentioned before for ssDNA, we obtain a⁽⁵⁰⁾=5.1 and a⁽⁷⁰⁾=6.7. These predicted values are now too small (by about a factor of 2.5) and show that a FJC model is not a sufficient model for the drag-tags. We thus need to take into account the stiffness of the drag-tags as well.

### EXAMPLE 8

### Estimating a: Stiff Polymers—Simple Theory

Again, we will apply our generalization of Teraoka's theory to predict the a values of the drag-tags, but this time we will take into account the finite stiffness of the label using a simple, “0^{th} order” approximation. As derived in Section 3.1, the molecular properties of the linear drag-tag are bᵤ=0.43 nm and b_{Ku}=0.78 nm. For all practical purposes, a sufficiently long stiff polymer of contour length L can be considered as a FJC if we use b_{Ku} as the monomer size and N_{K}=L/b_{Ku} as the number of monomers. Therefore, a simple way to take into account the finite flexibility of the drag-tag segments (backbone and arms) is to use Eq. (8) with the renormalized values

$${N}_{1} -> {N}_{1} \frac{{b}_{u}}{{b}_{Ku}} , {N}_{b} -> {N}_{b} \frac{{b}_{u}}{{b}_{Ku}} and {N}_{b}^{′} -> {N}_{b}^{′} \frac{{b}_{u}}{{b}_{Ku}} ,$$

while the number of arms a is kept constant and the monomer size is increased to b_{Ku}. The calculations are straightforward and we now obtain R_{H}⁽⁵⁰⁾=0.95 nm and R_{H}⁽⁷⁰⁾=1.10 nm for the tetramer and octamer labels, respectively. The corresponding alpha values are then calculated using Eq. (13), and we obtain a⁽⁵⁰⁾=9.44 and a⁽⁷⁰⁾=12.7. These predicted values are in much better agreement with the experiments than the results derived in Section 4.3, but we still under-predict the actual value of a by about 40%.

Several reasons for this discrepancy can be proposed. For instance, our 0^{th} order approach to the drag-tag stiffness, described in this subsection, is strictly valid only for very long polymer segments, which is not the case here since N_{b} and N₁ are rather small (our approach actually underestimates the effect of stiffness). This critical weakness of the theory presented so far will be examined in Section 5. Other effects, neglected in this paper, will be discussed in Section 7.

### EXAMPLE 9

### Branched Worm-like Chain Polymers

In this and subsequent examples the inventors improve upon the approach presented in Example 8 to take into account the stiffness of the label in a more appropriate way. First and foremost we note that Eq. (5), which gives the equation for the hydrodynamic radius of a macromolecule, can be easily calculated for any given branching architecture if the average inverse distance [symbol] Rᵢⱼ⁻¹ [symbol] between any two monomers i and j is known. In absence of excluded volume interactions, any two monomers i and j are in fact the end monomers of a linear chain. Therefore, an improved theory starts necessarily with a better approximation for the average [symbol] Rᵢⱼ⁻¹ [symbol] , which means a better approximation for the end-to-end distance of a linear worm-like polymer chain. We discuss this subject in Section 5.1. However, we do note that along the linear chain starting at the i^{th} monomer and ending at the j^{th} monomer, there might be side-chains and grafting points; such junctions may obviously have an impact on the usual chain statistics. This problem is discussed below.

We begin by reviewing the classical theory of the Kratky-Porod chain model for a linear worm-like polymer (the backbone), and then expand this theory to allow for the presence of side-chains attached to the linear chain.

### EXAMPLE 10

### The Worm-Like Chain Model for a Linear Chain

The average squared end-to-end distance of a polymer chain made of N segments can be written as following [14]:

$$〈 {R}^{2} 〉 = {\sum }_{i = 1}^{N} {\sum }_{j = 1}^{N} 〈 {\vec{r}}_{i} \cdot {\vec{r}}_{j} 〉 = {b}^{2} {\sum }_{i = 1}^{N} {\sum }_{j = 1}^{N} 〈 cos {θ}_{ij} 〉 \qquad ( 14 )$$

where \vec{r}ᵢ and \vec{r}ⱼ are bond vectors, b is the bond length (which is assumed constant), cosθᵢⱼ≅\vec{r}ᵢ·\vec{r}ⱼ/b² is the bond angle, and the average is taken over all possible chain conformations.

To account for the limited flexibility of real polymers we can assume that the bond angle between any two consecutive segments is only allowed to freely rotate, white its average value θ is maintained constant. This is the well-known Kratky-Porod model, or the worm-like chain (WLC) model. The average angle between any two arbitrary segments i and j can thus be written as follows:

$$〈 cos {θ}_{ij} 〉 = {( cos θ )}^{\vert j - i \vert} = {ⅇ}^{- \frac{\vert j - i \vert b}{{l}_{p}}} \qquad ( 15 )$$

where lₚ is the persistence length of the chain (note that the Kuhn length b_{K} of the chain is defined as being equal to 2lₚ). Using Eqs. (14) and (15), and changing the summation over bonds into an integral over the contour length of the chain, the mean square end-to-end distance [symbol] R² [symbol] can be rewritten as follows:

$$〈 {R}^{2} 〉 = {b}^{2} {\sum }_{i = 1}^{N} {\sum }_{j = 1}^{N} {( cos θ )}^{\vert j - i \vert} = 2 {R}_{max} {l}_{p} - 2 {l}_{p}^{2} [ 1 - exp ( - \frac{{R}_{max}}{{l}_{p}} ) ] \qquad ( 16 )$$

where Rₘₐₓ=Nb is the maximum end-to-end distance of the actual polymer (or the chain's contour length). The two well-known limits of Eq. (16) are the ideal FJC limit [symbol] R² [symbol] ≈b_{K}Rₘₐₓ, which applies when Rₘₐₓ>>lₚ, and the rod-like limit [symbol] R² [symbol] ≈Rₘₐₓ², valid when Rₘₐₓ<<lₚ. We used the result of the first limit in Section 4.4; however, our chains are really in the intermediate regime where the approximation [symbol] R² [symbol] ≈b_{K}Rₘₐₓ underestimates the mean chain end-to-end distance.

In order to use this chain model to calculate the hydrodynamic radius of a branched polymer, we have to compute the inverse end-to-end distance between any two monomers i and j. From Eq. (16), we can write:

$$〈 {R}_{ij}^{- 1} 〉 ≅ \frac{1}{\sqrt{2 \vert j - i \vert {bl}_{p} - 2 {l}_{p}^{2} ( 1 - exp [ - \frac{\vert j - i \vert b}{{l}_{p}} ] )}} \qquad ( 17 )$$

Together with a knowledge of the properties of the branching points, Eqs. (17) and (5) allow us to compute the hydrodynamic radius of branched drag-tags.

### EXAMPLE 11

### Estimating a: WLC Polymers—Simple Theory

First, we disregard the branching points and consider that the sequence of monomers between monomers i and j always forms a continuous WLC satisfying Eq. (17). This is the simplest way to improve upon Section 4.4. The details of the calculations are presented in Appendix A.

Using these equations it is possible to obtain a new numerical estimate for the effective friction coefficient of the two labels studied by Haynes et al.; we obtain R_{H}⁽⁵⁰⁾=1.11 nm and R_{H}⁽⁷⁰⁾=1.24 nm for the tetramer and octamer labels, respectively. From Eq. (13), the corresponding a-values are a⁽⁵⁰⁾=12.92 and a⁽⁷⁰⁾⁼16.04. This is a major improvement upon the results obtained previously. This simple calculation demonstrates very clearly the importance of taking into account the stiffness of the drag-tag molecule. We note that the persistence length of the labels has been taken as lₚ=½b_{Ku}=0.39 nm (see Section 3.1).

### EXAMPLE 12

### WLC Theory for Branched Labels

To properly calculate the hydrodynamic radius of branched polymers we need to evaluate the distance between any two monomers. When there is no branching point between the two monomers, Eq. (16) can be used. We propose here to improve the Kratky-Porod equation (16) and derive an expression for the end-to-end distance between two monomers in the case where we have branching points between them.

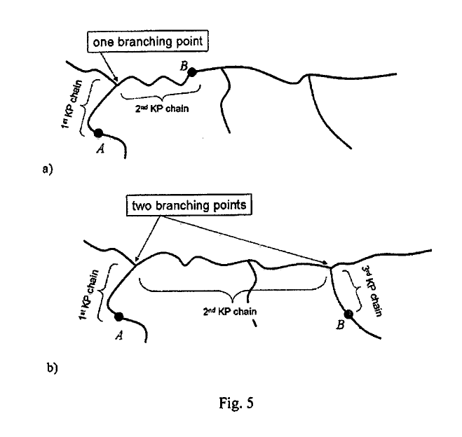

We start with a description of the branching points. In FIG. 5(a) we have a single branching point between the A and the B monomers. In FIG. 5(b) we have two branching points; note that the point where the middle arm is connected is not considered important since it is assumed that lateral arm does not disturb the KP nature of the backbone.

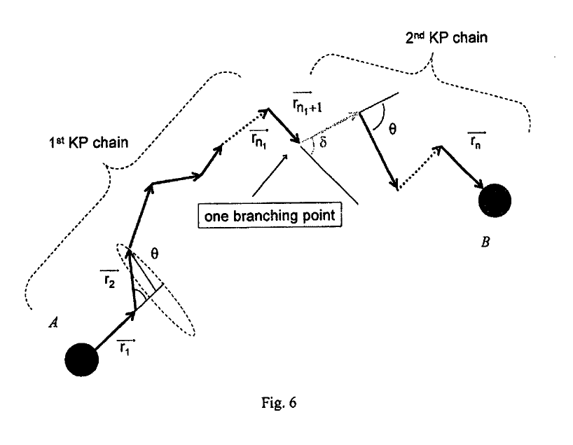

We assume that the linear polymer starting at the A monomer and ending at the B monomer is made of independent Kratky-Porod segments linked together at the branching points. For the case of a single given branching point, FIG. 6 shows in detail how we join together the two KP chains. As before, we assume that both KP chains have the same persistence length. The bond angle that makes the connection is simply assumed to be a freely-rotating bond angle δ, different from the average value θ.

The average [symbol] \vec{r}ᵢ·\vec{r}ⱼ [symbol] in Eq. (14) must be calculated differently if we have branching points. If there is one branching point between the i^{th} and j^{th} bond vectors (FIG. 5), we have: [symbol]
\vec{r}ᵢ·\vec{r}ⱼ=b²(cosθ)^{|j-i-1|}_{cosδ} (18)
where δ is the angle between the last bond vector of the 1^{st} KP chain and the first bond vector of the 2^{nd} KP chain. Similarly, if there are two branching points we use the expression: [symbol]
\vec{r}ᵢ·\vec{r}ⱼ=b²(cosθ)^{|j-i-2|}cos²δ (19)

We derive now the mean square end-to-end distance of a linear chain with one or two branching points. For just one such connection, found at monomer n₁ (see FIG. 5), we can rewrite Eq. (16) such as to put into evidence the angle δ:

$$〈 {R}_{ij}^{2} 〉 = {b}^{2} + {\sum }_{i = 1}^{{n}_{1}} {\sum }_{j = 1}^{{n}_{1}} 〈 {\vec{r}}_{i} \cdot {\vec{r}}_{j} 〉 + {\sum }_{i = {n}_{1} + 2}^{n} {\sum }_{j = {n}_{1} + 2}^{n} 〈 {\vec{r}}_{i} \cdot {\vec{r}}_{j} 〉 + 2 {\sum }_{i = 1}^{{n}_{1}} 〈 {\vec{r}}_{{n}_{1} + 1} \cdot {\vec{r}}_{i} 〉 + 2 {\sum }_{i = {n}_{1} + 2}^{n} 〈 {\vec{r}}_{{n}_{1} + 1} \cdot {\vec{r}}_{i} 〉 + 2 {\sum }_{i = {n}_{1} + 2}^{n} {\sum }_{j = 1}^{{n}_{1}} 〈 {\vec{r}}_{i} \cdot {\vec{r}}_{j} 〉 \qquad ( 20 )$$

The 2^{nd} and 3^{rd} terms in Eq. 20 are the statistical properties of the 1^{st} KP and the 2^{nd} KP chains, while the 4^{th} and the 5^{th} terms are the projections of the 1^{st} KP and the 2^{nd} KP chains onto the bond vector \vec{r}ₙ_{₁}₊₁. Using the notations

$${\vec{R}}_{1} = ( {\sum }_{i = 1}^{{n}_{1}} {\vec{r}}_{i} ) , \\ {\vec{R}}_{2} = ( {\sum }_{i = {n}_{1} + 2}^{n} {\vec{r}}_{i} ) \qquad ( 21 )$$

for the two KP sub-chains, we obtain:

$$〈 {R}^{2} 〉 = {b}^{2} + 〈 {R}_{1}^{2} 〉 + 〈 {R}_{2}^{2} 〉 + 2 〈 {\vec{r}}_{{n}_{1} + 1} \cdot ( {\vec{R}}_{1} + {\vec{R}}_{2} ) 〉 + 2 cos δ {\sum }_{i = {n}_{1} + 2}^{n} {\sum }_{j = 1}^{{n}_{1}} {{b}^{2} ( cos θ )}^{\vert j - i - 1 \vert} \qquad ( 22 )$$

For a linear chain with two branching points we obtain a similar result:

$$〈 {R}^{2} 〉 = 2 {b}^{2} + 〈 {R}_{1}^{2} 〉 + 〈 {R}_{2}^{2} 〉 + 〈 {R}_{3}^{2} 〉 + 2 〈 {\overline{r}}_{{n}_{1} + 1} \cdot ( {\vec{R}}_{1} + {\vec{R}}_{2} + {\vec{R}}_{3} ) 〉 + 2 〈 {\overline{r}}_{{n}_{1} + 1} \cdot ( {\vec{R}}_{1} + {\vec{R}}_{2} + {\vec{R}}_{3} ) 〉 + 2 \vert cos δ \vert {\sum }_{i = {n}_{1} + 2}^{{n}_{2}} {\sum }_{j = 1}^{{n}_{1}} {{b}^{2} ( cos θ )}^{\vert j - i - 1 \vert} + 2 {cos}^{2} δ {\sum }_{i = {n}_{1} + 2}^{n} {\sum }_{j = 1}^{{n}_{1}} {{b}^{2} ( cos θ )}^{\vert j - i - 1 \vert} + 2 cos δ {\sum }_{i = {n}_{1} + 2}^{{n}_{2}} {\sum }_{j = {n}_{2} + 2}^{n} {{b}^{2} ( cos θ )}^{\vert j - i - 1 \vert} \qquad ( 23 )$$

where \vec{r}ₙ_{₂}₊₁ denotes the first bond vector of the second KP chain, and

$${\vec{R}}_{2} = ( {\sum }_{i = {n}_{1} + 2}^{{n}_{2}} {\vec{r}}_{i} ) , \\ {\vec{R}}_{3} = ( {\sum }_{i = {n}_{2} + 2}^{n} {\vec{r}}_{i} ) \qquad ( 24 )$$

If we assume that δ=θ, i.e. there are no branching points along linear chains, or equivalently all the bond angles are the same, both Eqs. (20) and (23) reduce to Eq. (16). However, if we assume that δ≠θ we obtain chains with larger or smaller hydrodynamic radii. Together with Eq. (5), these equations allow us to compute the hydrodynamic radius of any type of branched drag-tag. The end result will now be a function of the angle δ.

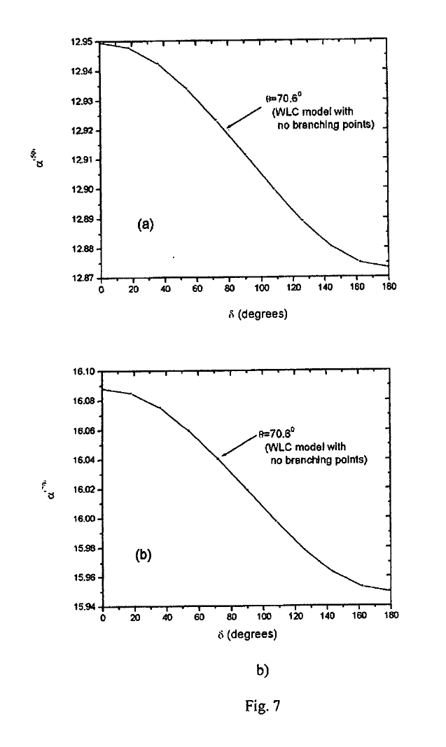

We show in FIG. 7 the predictions of this model for the a values of the tetramer and octamer branched labels. Although the range of angle δ is taken from 0 to 180°, it is clear that not all of these values are meaningful because of geometrical constraints. The result clearly indicates that the value of the branching angle δ is not an important parameter for these labels. Consequently, we will assume a uniform bond angle δ=θ=70.6° (corresponding to lₚ=½b_{Ku}=0.39 nm, see Eq. 15) in the next section.

### EXAMPLE 13

### Optimizations of the Branched Labels, Comparing Linear and Branched Labels

The problem of optimising the architecture of an ELFSE label cannot be approached solely by an experimental trial-and-error method because of the difficulties in the chemical synthesis of large macromolecules. Moreover, these drag-tags, either linear or branched, must have very specific properties—uncharged, hydrophilic, monodisperse, etc. It is therefore essential to find design principles for the optimal type of branching which would provide the largest effective friction coefficient a for a given number of monomers.

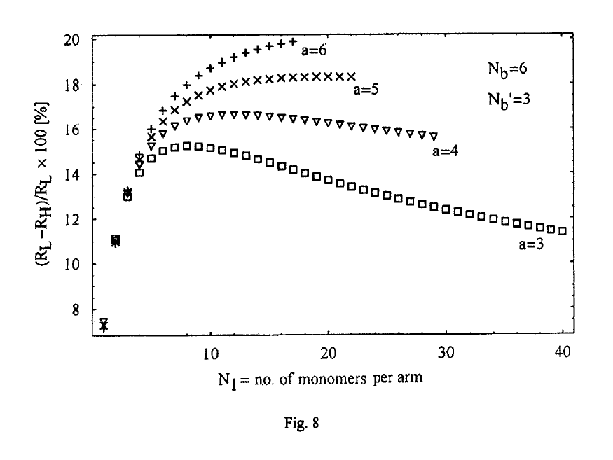

We first examine the hydrodynamic radii of a linear polymer and of a branched polymer (with the architecture shown in FIG. 3), both with the same total number of monomers N. In the next section, we will compare the experimental data for the tetramer and octamer labels with the optimal parameters found for this type of branching. Since we are keeping N fixed, the optimal architecture will be found by successive rearrangements of the monomers. We use the ratio (R_{L}−R_{H})/R_{L} to compare the hydrodynamic radius R_{L} of a linear label with the hydrodynamic radius R_{H} provided by a branched label, both with the same number N=a(N_{b}+N₁)+2N_{b}′−N_{b} of monomers. We present in FIG. 8 the results using the freely jointed chain (FJC) model of Section 4.3. For a backbone with a fixed size (a−1)N_{b}+2N_{b}′ we investigate the influence of the length N₁ of the side chains on the ratio (R_{L}−R_{H})/R_{L}. We chose several values for the number of arms a and we kept the distance between consecutive side chains constant at N_{b}=6.

From FIG. 8 we note that (R_{L}−R_{H})/R_{L}>0 for all values of N₁ investigated, which means the drag provided by the linear label is always higher than that provided by a branched label. The reason for this is that with a fixed-length backbone, a branched polymer is essentially a compact star polymer with a small radius of gyration. Indeed, as the number of arms increases, the branched polymer becomes even more compact and less favourable for ELFSE.

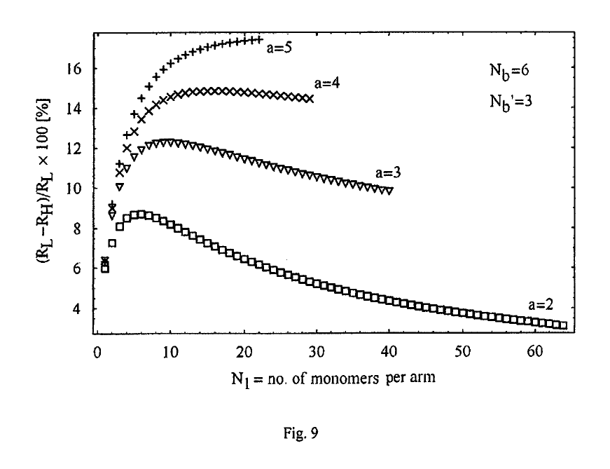

A somewhat similar situation is encountered if the stiffness of the polymer is taken into account. FIG. 9 shows the results from the WLC model. The general aspect of the curves seen in FIG. 8 is maintained, except that the curves are lower by a few percents; this decrease is due to the stiffness of the chains. The a=2 case is essentially a linear polymer since the two branches are located very close to the two ends of the backbone section. Again, a linear polymer provides more friction for the same real estate.

Quantitatively, our results explain the somewhat surprising data in Table 1. The fact that the effective friction coefficient a increases almost linearly with the total molecular size of the branched labels is actually due to the fact that the arms are rather short. The situation would be quite different for long arms, as we shall see in the next section.

Although linear polymers are preferable, branched polymers offer practical advantages because of the possibility of synthesizing larger monodisperse molecules in a simple, stepwise way. Our results suggest two branching strategies. First, adding two very long arms near the ends of the backbone molecule can add a large amount of friction with very little loss when compared to having all the monomers forming a single linear chain (see bottom curve in FIG. 9). Second, a large number of short arms can also achieve a similar result: in fact, FIG. 9 tells us that if the arms are shorter in size than the distance between them along the backbone (N₁<N_{b}), the value of a is comparable to that we would obtain with a linear polymer. In the latter case, however, we would also benefit from an effect that is not included in our models: the persistence length of the backbone of a branched molecule actually increases [15] when a large number of such short arms are present.

### EXAMPLE 14

### Optimization of the Branched Labels, Optimal Architectures for the Tetramer and Octamer Labels

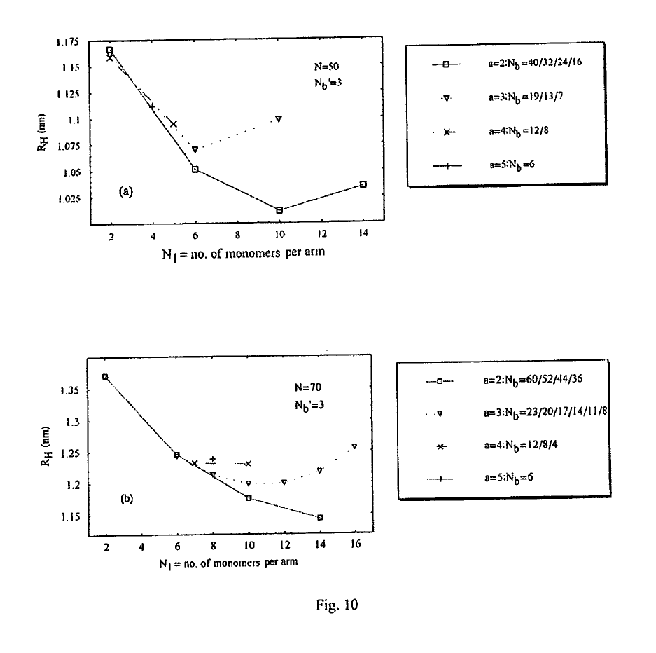

We now compare the hydrodynamic radii of the tetramer and octamer labels (as derived from the experimental data) with the hydrodynamic radii predicted for an optimal label of the same type of branching. Again, we keep the total number of monomers N fixed at either 50 (tetramer) or 70 (octamer). We use the persistence length lₚ=0.39 nm, determined in Example 2, and the WLC model presented in Example 11 (i.e., we assume that the branching angle δ=θ since we showed in Example 12 that its value has very little impact on the final result). With these numbers and the relevant equations, it is possible to compute the hydrodynamic radius of all possible combinations giving the same value of N when only N_{b}′ is kept fixed. The results are shown in FIG. 10.

The curves show two interesting regimes, already mentioned in Example 13. First, the largest hydrodynamic radii are obtained on the left when we have only two short arms (a=2, N=2). This is not surprising because we already know that the maximum value of R_{H} is always found for the linear polymer (a=0). For the 50-mer label, the best set of parameters (N₁=2, a=2 and N_{b}=40—FIG. 10a) gives an effective friction coefficient a⁽⁵⁰⁾=14.24, which is 14% larger than obtained experimentally with 5 arms (see Table 1). Similarly, the optimal 70-mer label (N₁=2, a=2 and N_{b}=60—FIG. 10b) has an effective friction coefficient of a⁽⁷⁰⁾=19.65., a 20% improvement. These are excellent improvements in themselves and very close to the maximum values obtained for linear chains which would give a⁽⁵⁰⁾=16.83 and a⁽⁷⁰⁾=22.42). However, since long linear chains are difficult to synthesize, this strategy is not likely to be useful in practice.

On the other hand, we see that some of the curves are going up for large values of N₁ (i.e., for long arms). This corresponds to the second case mentioned before: a few long arms, preferably situated near the ends of the molecule, also provide a quasi-linear chain with a potentially large drag coefficient. In the N=70 case, for instance, a 3-branch polymer with N₁=16 monomers per branch (a hexamer) and a distance of N_{b}=8 monomers between the arms would have produce a slightly higher friction coefficient than the octamer used experimentally (a⁽⁷⁰⁾=16.49 vs a⁽⁷⁰⁾=16.04). Obviously, the curves would go up even further for larger values of N. Although the values obtained with this strategy are slightly lower than those obtained with the first strategy, this is a much better approach since it avoids the synthesis of extremely long and monodisperse backbones. Instead, one can use moderately long building blocks, such as hexamers in this example, together with a moderately long backbone (30 mers in total) and a only a few branching points (2 or 3).

### EXAMPLE 15

### The Difference between the Two Primers

Table 1 shows an interesting effect when branched labels are used: the apparent value of at appears to increase slightly when a larger DNA primer is used to pull it through the electrophoretic system. This effect is of order 10% for the tetramer and 5% for the octamer. However, no such effect was reported for the linear labels of size N=30. We suspect that at least two different phenomena can possibly explain this second-order effect, and it is not possible to distinguish between them with the current state of the theory and the very restricted amount of experimental data presently available.

First, it is known that there are corrections to the electrophoretic mobilities related to the end effects [8,9]; for linear labels it has been shown that this may slightly increase the apparent friction coefficient of a drag-tag as the size of the DNA increases. Unfortunately, there is no end-effect theory that would apply to branched labels, and therefore no further quantitative insight can be gained in this direction.

Second, the fact that the branched drag-tags are bulky may also induce some steric segregation between the DNA and the labels; the standard ELFSE theory used here assumes that the hybrid molecule can be treated as a coherent sequence of blobs forming a single random coil. The case of the segregated label has yet to be studied theoretically [11], but it is likely that hybrid molecules would segregate to different extents for different molecular sizes. Segregation is also directly related to excluded volume interactions and electrostatic interactions, effects that we did not consider in this study.

### EXAMPLE 16

### Conclusions

Since it is quite difficult to produce long, monodisperse linear polymer chains to be used as drag-tags for ELFSE, Haynes et al. recently proposed to build branched drag-tags from various monodisperse building blocks (shorter linear chains that can be attached together). Since a branched object is necessarily more compact, one would instinctively conclude that this approach would lose in terms of performance although it may gain in ease of preparation. Surprisingly, the experimental results of Haynes et al. actually showed an almost linear increase in the value of the effective drag coefficient ax with the molecular weight of the label.

In this application the inventors present three models for uniform comb-like branched polymers: the FJC model, the worm-like chain model, and finally a modified WLC model that took into account the properties of the branching points. For all three models, the underling theory used to calculate the friction properties has been based on the work of Teraoka [10]. Comparing the predictions of these three models with the measured a values, we saw that the FJC model gave values about 50% lower than that of the experiment, while for the WLC model the agreement was within a few percent (the modified WLC provided little improvement). We also speculate that the small dependence of a upon the size of the DNA could be explained by two possible phenomena that we neglected in this paper (namely, end effects [8] and steric segregation).

Based on our results herein and on polymer theory, the inventors deduce three different approaches to optimizing the architecture of polymer labels for ELFSE:
First, since linear polymers always have a larger hydrodynamic radius than branched polymers having the same total number of monomers, one should always synthesize the longest possible linear backbones.
Because of the first point above, there is no point in having a large number of moderately long branches. However, a large number of short branches would help because it is known that this would increase the Kuhn length of the backbone (in effect, making a stiffer polymer with a fatter backbone). Previous theoretical investigations indicated that this effect would start to play a role when the length of the arms is comparable to the distance between them (N₁≅N_{b}). The cases N₁<<N_{b} (arms too short to stiffen that backbone because they do not interact sterically with each other) and N₁>>N_{b} (arms uselessly long) may be avoided, although longer arms may present some advantages.
We found that a few (preferably 2) long arms attached near the end of the molecule would be an excellent way to build efficient drag tags since such molecules resemble linear chains while being made of monodisperse building blocks.
   We thus conclude that the approach used by Haynes et at. can indeed produce superb ELFSE drag-tag if the design follows the guiding principles given above.

### EXAMPLE 17

In calculating the hydrodynamic radius of a branched polymer we follow the formalism of Teraoka [10], except that now the pre-averages are calculated using Eq. (17). The hydrodynamic radius is written as follows: [symbol]
R_{H}⁻¹=C_{A}u_{A}(N₁,lₚ)+C_{B}u_{B}(N₁,a,lₚ)+C_{C}u_{C}(N_{b},N_{b}′,a,lₚ)+C_{D}u_{D}(N₁, N_{b},N_{b}′,a,lₚ) (A1)
where the coefficients C_{A}, C_{B}, C_{C}, and C_{D} were defined in Section 4.2, and the functions u_{A} through u_{D} are given by:

$$\begin{matrix}{u}_{A} ( {N}_{1} , {l}_{p} ) = {( \frac{6}{π} )}^{1 / 2} \frac{2}{{N}_{1}^{2}} {\int }_{0}^{{N}_{1} - 1} {\int }_{u + 1}^{{N}_{1}} {[ 2 \vert v - u \vert {bl}_{p} - 2 {l}_{p}^{2} ( 1 - exp ( - \vert v - u \vert b / {l}_{p} ) ) ]}^{- 1 / 2} ⅆ u ⅆ v & ( A2 ) \\ {u}_{B} ( {N}_{1} , {N}_{ b} , a , {l}_{ p} ) = {( \frac{6}{π} )}^{1 / 2} \frac{2}{{N}_{1}^{2}} {\sum }_{i = 1}^{a - 1} \frac{a - i}{a ( a - 1 )} \times \\ {\int }_{0}^{{N}_{1}} {\int }_{0}^{{N}_{1}} {[ 2 ( 2 {N}_{1} + ⅈ {N}_{b} - u - v ) {bl}_{p} - \\ 2 {l}_{p}^{2} ( 1 - exp ( - ( 2 {N}_{1} + ⅈ {N}_{b} - u - v ) b / {l}_{p} ) ) ]}^{- 1 / 2} ⅆ u ⅆ v & ( A3 ) \\ {u}_{C} ( {N}_{b} , a , {l}_{p} ) = {( \frac{6}{π} )}^{1 / 2} \frac{2}{{( ( a + 1 ) {N}_{b} )}^{2}} {\int }_{0}^{( a - 1 ) {N}_{b} + 2 {N}_{b}^{′} - 1} \\ \{ {\int }_{0}^{( a - 1 ) {N}_{b} + 2 {N}_{b}^{′} - 1} {[ \begin{matrix}2 \vert v - u \vert {bl}_{p} - \\ 2 {l}_{p}^{2} ( \begin{matrix}1 - \\ exp ( - \vert v - u \vert b / {l}_{p} )\end{matrix} )\end{matrix} ]}^{- 1 / 2} \} ⅆ u ⅆ v & ( A4 ) \\ {u}_{D} ( {N}_{1} , {N}_{b} , a , {l}_{p} ) = {( \frac{6}{π} )}^{1 / 2} \frac{2}{{aN}_{1} ( ( a - 1 ) {N}_{b} + 2 {N}_{b}^{′} )} \times \{ \begin{matrix}{\sum }_{l = 1}^{a - 1} {\int }_{1}^{{iN}_{b}} {\int }_{0}^{{N}_{1}} {[ \begin{matrix}2 ( v + u ) {bl}_{p} - \\ 2 {l}_{p}^{2} ( 1 - exp ( - ( v + u ) b / {l}_{p} ) )\end{matrix} ]}^{- 1 / 2} + \\ {\int }_{0}^{( a - 1 ) {N}_{b} + 2 {N}_{b}^{′}} {\int }_{0}^{{N}_{1}} {[ \begin{matrix}2 ( v + u ) {bl}_{b} - \\ 2 {l}_{p}^{2} ( 1 - exp ( - ( v + u ) b / {l}_{p} ) )\end{matrix} ]}^{- 1 / 2}\end{matrix} \} & ( A5 )\end{matrix}$$

where b=0.43 nm is the monomer size of the label.

### SUMMARY OF SELECTED PREFERRED EMBODIMENTS OF THE INVENTION

The present invention, at least in selected embodiments, provides design principles for branched polymers for use as polymeric end labels (or drag tags) in End-Labeled Free Solution Electrophoresis (ELFSE) of DNA. The optimal branching provides high potential frictional drag for a given number of monomers (or molecular weight). The invention also provides design principles for the design of cationic labels that have an increased effective frictional drag effect for ELFSE.

Deduced approaches towards optimizing the architecture and composition of polymer labels for ELFSE in accordance with the teachings of the present invention:

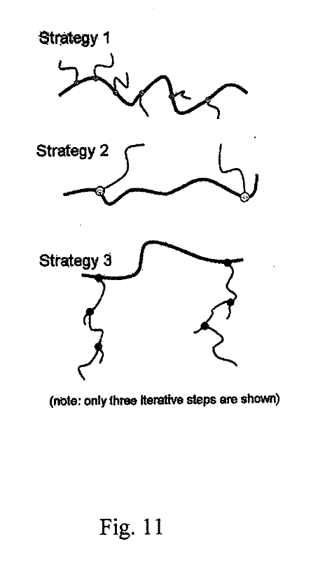

1. Since linear polymers always have a larger hydrodynamic radius than branched polymers having the same total number of monomers, the longest possible backbone should preferably be synthesized.
2. Hence there is less incentive to design an end label having a large number of moderately long branches. Instead, short branches should preferably be used to increase the Kuhn length of the backbone (making a stiffer polymer with a thicker backbone), whereby the distance between the branching points should be approximately equal to or less than the length of the branches themselves. (N_{b}≅N₁). This is illustrated schematically in FIG. 11a;
3. Alternatively, a few long branch arms attached near the end of the molecule would also be an excellent way to build efficient drag tags since such molecules resemble linear chains while being made of monodisperse building blocks. This is schematically illustrated in FIGS. 11b and 11c, with FIG. 11c further illustrating iterative branching of the branch arms.

The hydrodynamic radii of the linear and branched labels (all neutral) were calculated using standard models like the freely jointed chain model (FJC) and the Kratky-Porod worm like chain model (WLC). Based on comparisons of the theory with the experimental data, the inventors propose that the design of new branched labels should use either side chains whose length is comparable to or greater than the distance between the branching points, or longer branches (preferably two longer branches) located near the ends of the molecule's backbone. The theoretical calculations were based on three major models for branched polymers: 1. The FJC, the WLC, and a modified WLC that takes into account the properties of the branching points. The first of these models is based on the work of Teraoka while the others are new theories put forward by the authors. Comparing the predictions of these three models with the experimental results, it was determined that the FJC model under-predicted the friction values by 50%. The WLC model and the modified WLC model provided close agreement to the experimental results.

Hydrodynamic Radii for Branched Freely Jointed Chains

The method is based on a constrained optimization procedure for the hydrodynamic radii of banched labels. The total number of monomers N=a(N₁+N_{b})+2N_{b}′−N_{b} is kept constant and all the other parameters are varied. This means a—the number of arms along the backbone, N₁—the number of bonds on each side chain, N_{b}—the number of bonds between the branching points along the main backbone, N_{b}′—the number of bonds at each of the two ends of the molecule. The inventors selected those parameters that give high hydrodynamic friction.

Estimating a: WLC Polymers

The unsatisfying results obtained with the FJC indicated that the stiffness of the drag-tag molecule must be taken into account in a proper way. Simply rescaling the number of monomers by arranging them into equivalent Kuhn blobs is not sufficient. Application of the theory for branched labels: To properly calculate the hydrodynamic radius of branched polymers the distance between any two monomers has been carefully considered. Derivation of a Kratky-Porod-like equation led to a new expression for the end-to-end distance between two monomers in the case where branching points between them exist. Therefore, based on comparisons of the theory with the experimental data, the design of new branched labels should use either side chains whose length is comparable to or greater than the distance between the branching points or longer branches (preferably two longer branches) located near the ends of the molecule's backbone for optimized separation. In the latter case, we further suggest that the process can be used iteratively, i.e., a single branching point near the other end of each branch can be added, and a new branch attached at that position. This process can in principle be continued until the desired value of alpha is reached (for example see FIG. 11c).

While the invention has been described with reference to particular preferred embodiments thereof, it will be apparent to those skilled in the art upon a reading and understanding of the foregoing that numerous methods for polymer molecule modification and separation, as well as corresponding end labels for their separation, other than the specific embodiments illustrated are attainable, which nonetheless lie within the spirit and scope of the present invention. It is intended to include all such methods and apparatuses, and equivalents thereof within the scope of the appended claims.

### REFERENCES

[1] Viovy, J. L., Rev. Mod. Phys. 2000, 72, 813-872.
[2] Meagher, R., J., Won, J. I., McCormick, L. C. et al., Electrophoresis 2005, 26, 331-350.
[3] Ren, H., Karger, A., Oaks, F., Menchen, S., et al., Electrophoresis 1999, 20, 2501.
[4] Mayer, P., Slater, G. W., Drouin, G., Anal. Chem. 1994, 66, 1777-1780.
[5] Sudor, J., Novotny, M. V., Anal. Chem. 1995, 67, 4205-4209.
[6] Heller, C., Slater, G. W., Mayer, P., Dovichi, N., et al., J Chrom A 1998, 806, 113-121.
[7] Vreeland, W. N., Desiuisseaux, C., Karger, A. E., Drouin, G., et al. Anal. Chem. 2001, 73, 1795-1803.
[8] McCormick, L. C, Slater, G. W., Karger, A. E., Vreeland, et al., J Chrom. A 2001, 924,43-52.
[9] McCormick, L. C., Slater, G. W., Electrophoresis 2005, 26, 1659-1667.
[10] Teraoka, I, Macromolecules 2004, 37, 6632-6639.
[11] Long, D., Ajdari, A., Electrophoresis 1996, 17, 1161-1166.
[12] Long, D., Viovy, J. L., Ajdari, A., J Phys.:Condens. Matter 1996, 8, 9471-9475.
[13] Long, D., Dobrynin, A. V., Rubinstein, M., Ajdari, A., J. Chem. Phys. 1998, 108, 1234-1244.
[14] Doi, M.; Edwards, S. F., The theory of polymer dynamics, Clarendon Press, Oxford 1986.
[15] Gauger, A.; Pakula, T. Macromolecules 1995, 28, 190-196.

1. An end label suitable for attachment at or near to an end of a polymer molecule, so as to increase the hydrodynamic drag of the polymer molecule during motion through a liquid substance such as during electrophoresis, with or without the presence of an electroosmotic flow, the end label comprising:
(1) a backbone;
(2) at least one branch arm extending from the backbone at branch point(s) therein, the branch arm(s) selected from at least one of the group consisting of:
(2a) a plurality of branch arms each being substantially shorter than the backbone and having a length about equal to or greater than a length of the substantially linear backbone between adjacent consecutive branch points; and
(2b) at least one branch arm each extending from a corresponding branch point at or near at least one end of the linear backbone, each branch arm optionally including further iterative branching extending from a free end thereof.

2. The end label of claim 1, wherein the substantially linear backbone and each branch arm each comprise monomer units.

3. The end label of claim 2, wherein the end label comprises from 30-500 monomer units.

4. The end label of claim 2, wherein end label is a polypeptide and/or polypeptoid, and the monomer units comprises natural and/or non-natural amino acids.

5. The end label of claim 1, wherein the at least one branch arm each extending from a corresponding branch point at or near at least one end of the linear backbone, comprises two branch arms each extending from an opposite end of the substantially linear backbone.

6. The end label of claim 1, wherein the backbone comprises from 20-10000 monomer units, and the branch arms comprise from 2-1000 branch arms each comprising from 5-10000 monomer units.

7. The end label of claim 6, wherein the branch arms (2a) comprise from 2-1000 branch arms each comprising from 5-1 0000 monomer units.

8. The end label of claim 6, wherein the branch arms (2b) comprise from 1-1000 branch arms each comprising from 5-10000 monomer units.

9. The end label of claim 1, wherein the plurality of branch arms extending from the linear backbone at branch points therein are substantially equally spaced along the linear backbone, each branch arm having a length substantially equal to every other branch arm, and substantially equal to a length of said linear backbone between consecutive branch points.

10. The end label of claim 1, said at least one branch arm each extending from a corresponding branch point at or near at least one end of the linear backbone, each including iterative branching comprising at least two further branch arm extensions to each branch arm, each extension extending at or near an end of each previous extension closer to the substantially linear backbone.

11. A method for constructing an end label for attachment to a polymer molecule to increase the hydrodynamic drag of the molecule through a liquid such as during electrophoresis or electroosmotic flow, the method comprising the steps of:
(1) synthesizing a substantially linear backbone comprising a plurality of monomer units; and
(2) synthesizing at least one branch arm extending from the backbone at branch point(s) therein, the branch arm(s) selected from at least one of the group consisting of:
(2a) a plurality of branch arms each being substantially shorter than the backbone and having a length about equal to or greater than a length of the substantially linear backbone between adjacent consecutive branch points; and
(2b) at least one branch arm each extending from a corresponding branch point at or near at least one end of the linear backbone, each branch arm optionally including further iterative branching extending from a free end thereof.

12. The method of claim 11, wherein the substantially linear backbone and the branch arms comprise monomer units, and the monomer units are natural and/or unnatural amino acids, said end label comprising a polypeptide and/or a polypeptoid.

13. A plurality of covalently modified polymer molecules having more than one length, suitable for separation via ELFSE, each comprising a substantially linear sequence of monomer units, and having covalently attached to at least one end thereof an end label of claim 1.

14. The plurality of polymer molecules of claim 13, each comprising ssDNA, derived from at least one DNA sequencing reaction.

15. A method for sequencing a section of a DNA molecule, the method comprising the steps of:
(a) synthesizing a first plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific adenine base in said section of DNA;
(b) synthesizing a second plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific cytosine base in said section of DNA;
(c) synthesizing a third plurality of ssDNA molecules each comprising a sequence identical to at least a portion at or near the 5′end of said section of DNA, said ssDNA molecules having substantially identical 5′ ends but having variable lengths, the length of each ssDNA molecule corresponding to a specific guanine base in said section of DNA;
(d) synthesizing a fourth plurality of ssDNA molecules each comprising a sequence identical to at least a portion at or near the 5′end of said section of DNA, said ssDNA molecules having substantially identical 5′ ends but having variable lengths, the length of each ssDNA molecule corresponding to a specific thymine base in said section of DNA;
(e) attaching an end label of claim 1 at or near at least one end of said ssDNA molecules to generate end-labeled ssDNAs; and
(f) subjecting each plurality of end labelled ssDNA molecules to free-solution electrophoresis;
(g) identifying the nucleotide sequence of the section of DNA in accordance with the relative electrophoretic mobilities of the end labeled ssDNAs in each plurality of ssDNAs;
wherein any of steps (a), (b), (c), and (d) may be performed in any order or simultaneously;
whereby each end label imparts increased hydrodynamic friction to at least one end of each end-labeled ssDNAs thereby to facilitate separation of the end-labeled ssDNAs according to their electrophoretic mobility.

16. The method of claim 15, wherein the end labels are uncharged.

17. The method according to claim 15, wherein the section of DNA comprises less than 2000 nucleotides.

18. The method according to claim 17, wherein the section of DNA comprises less than 500 nucleotides.

19. The method according to claim 18, wherein the section of DNA comprises less than 100 nucleotides.

20. A DNA sequencing kit comprising the end label of claim 1, together with at least one other component for a DNA sequencing reaction.
